(19) United States
(12) Patent Application Publication
(10) Pub. No.: US 2012/0046724 A1
(43) Pub. Date: Feb. 23, 2012

(54) IMPLANTABLE CARDIAC STIMULATION DRUG RELEASING ELECTRODE

(76) Inventor: Anna Norlin Weissenrieder, Stockholm (SE)

(21) Appl. No.: 12/373,578

(22) PCT Filed: Jul. 13, 2006
(86) PCT No.: PCT/SE2006/000882

§ 371 Date: Dec. 1, 2009

(51) Int. Cl. A61N 1/05; H01R 43/00

(52) U.S. Cl. 607/120; 29/825

(57) ABSTRACT

In an implantable cardiac stimulation electrode, an implantable cardiac stimulation lead, and a stimulation delivery method, the electrode body is provided with a matrix metalloproteinase inhibitor or a precursor thereof, in a release structure that allows release of the metalloproteinase inhibitor or precursor thereof from the electrode body into surrounding tissue, after implantation.

### BACKGROUND OF THE INVENTION

1. Field of the Invention

The present invention relates to an implantable cardiac stimulation electrode, of the type configured for releasing a drug from said electrode, and to an implantable cardiac lead embodying such an electrode. The present invention further relates to a method for the production of structure for releasing such drug from an electrode and to a method of improving the biocompatibility of an electrode. The present invention also relates to modes of accomplishing a lowered chronic pacing treshold in the heart of a patient and of preventing or reducing formation of fibrous tissue in a patient.

2. Description of the Prior Art

The stimulation pulse of a pacemaker system is generated by a pulse generator and transferred to the heart by a cardiac lead. The pulse enters the heart via a cardiac stimulation electrode located at the end of the lead. To close the electric circuit, the stimulation pulse leaves the heart via a counter electrode which transfers it back to the pulse generator. The counter electrode may alternatively be the encapsulation of the pulse generator.

When a cardiac lead is implanted, the electrode will cause trauma on the nearby tissue. During the healing process, scar tissue forms around the electrode, encapsulating it from healthy heart tissue. This regenerated tissue (often called fibrous tissue or scar tissue) does not exhibit the ability to activate when subjected to electrical stimuli. It can, however, transfer the impulse to healthy heart cells by conduction. When fibrous tissue has been formed around the electrode, pulses of higher amplitude must be applied to the electrode to ensure activation of the healthy excitable myocardial cells. According to the law of electrical field strength, the field strength diminishes with the square of the distance, which highlight the importance of keeping the thickness of the fibrous tissue at a minimum, so that energy consumption remain low. For reduction of fibrous tissue thickness, design strategies such as surface topography modifications of the electrode to improve biocompatibility or steroid eluting electrodes to reduce initial inflammation have been developed.

If a sufficiently high electrical stimulus (current or potential disturbance) is applied to an implanted cardiac stimulation electrode, a critical number of heart cells are activated. The activation will spread to all other cells in the heart and contract the heart tissue. The lowest potential or current disturbance that causes contraction is called the pacing threshold. If the stimulus is lower than the treshold, no activation will be initiated.

When an electrode is implanted the pacing treshold will rise with time post implantation. This effect is due to the activation of defence mechanisms in the body when a foreign material is implanted. During the first weeks following implantation, fluids and biological species accumulate around the cardiac electrode as a result of inflammation. The changed environment around the electrode induces a well-documented increase of the pacing treshold in comparison to the initial, acute, treshold. With prolonged implantation time the treshold stabilises at a chronic level, provided the healing response proceeds normally. Due to the formation of a fibrous capsule (scar tissue) as a result of the said defence mechanisms, the chronic treshold is generally higher compared to the acute treshold. Steroid eluting electrodes have demonstrated the ability to reduce the initial inflammation and avoid the acute increase of treshold following implantation. The effect of steroid elution on chronic inflammation is debated. It has also been suggested to coat electrodes with a membrane, e.g. a Nafione® membrane, to lower the chronic pacing treshold.

The concept of coating of medical instruments in order to improve their biocompatibility may be illustrated by the teachings of J. Biomed. Mater. Res. 1998 May; 40(2) :264-274, which describes a method for depositing onto medical instruments highly biocompatible and bioactive surface coatings, of diamond-like carbon or metals, which can promote and stabilize cell attachment. These surfaces were further altered to either promote or inhibit cell growth by an additional overcoat of biological materials, including extracellular matrix proteins, laminin, fibronectin and collagen IV. Possible applications for pacemaker electrodes were discussed.

An example of modification of pacemaker electrodes is given in Biosens. Bioelectron. 1997;12(8) :853-865, which reports on chemical modification of metal electrodes in order to enhance biocompatibility or improve cell adhesion properties. The electrodes were modified with a thin polysiloxane network which allowed for further derivatization with a poly(ethylene glycol) layer. The primary goal was to suppress inflammatory response of tissue after implantation of electrodes.

WO 02/055121 discloses an intravascular stent having a coating comprising a crosslinked amphiphilic polymer and a sparingly water soluble matrix metallo-proteinase inhibitor (MMP inhibitor, MMPI). It is reported that preclinical and clinical results show good luminal areas and reduced intimal thickening.

WO 2004/056353 discloses local administration of a matrix metalloproteinase inhibitor, or a pharmaceutically acceptable salt thereof, optionally in conjunction with one or more active ingredients, and a device, typically a stent, adapted for such local administration. It is referred to a need for effective treatment and drug delivery systems for preventing and treating intimal thickening or restenosis that occur after injury, e.g. in heart.

WO 95/24921 discloses the use of an MMP inhibitor, especially a collagenase inhibitor, in the manufacture of a medicament for the treatment of a natural or artificial tissue comprising extracellular matrix components to inhibit contraction of the tissue and methods for the treatment of tissue comprising extracellular matrix components to inhibit contraction.

### SUMMARY OF THE INVENTION

An object of the present invention is to improve the biocompatibility of an implantable cardiac stimulation electrode.

Another object of the present invention is to prevent or reduce formation of fibrous tissue (scar tissue) in a patient, resulting from implantation of an implantable cardiac stimulation electrode. In other words, an object of the present invention is to lower the chronic pacing treshold in the heart of a patient using an implantable cardiac stimulation electrode.

Mechanical stress creates friction between tissue and an implant, which amplifies the trauma and impairs the healing mechanisms. A further object of the present invention is to reduce inflammation induced by mechanical stress, particularly on a long view or chronically.

The above mentioned objects, as well as further objects of the invention, which should be apparent to a person skilled in the art after having studied the description below, are achieved by the different aspects of the present invention as described herein.

According to a first aspect of the invention, there is provided an implantable cardiac stimulation electrode, having a structure for releasing a matrix metalloproteinase inhibitor or a precursor thereof from said electrode.

Thus, the inventive electrode allows for improved tissue healing by reduction of tissue destruction caused by mechanical stress between the electrode surface and the tissue, reduced risk of inflammation, and reduced thickness of the fibrous capsule formed around an implanted electrode. This would result in better incorporation of the electrode with the cardiac tissue and allow for reduced energy requirements during pacing. In other words, the consequences of tissue irritation caused by implantantion of a cardiac electrode will be diminished by the present invention. The present invention provides for a balanced healing process after implantantion of a cardiac electrode.

Herein, the terms “matrix metalloproteinase inhibitor”, “MMP inhibitor” and “MMPI” will be used interchangeably with a similar meaning.

According to one embodiment, said structure for releasing a matrix metalloproteinase inhibitor or a precursor thereof from said electrode is a coating of a carrier composition on at least a portion of the surface of the electrode. This carrier composition is composed of the matrix metalloproteinase inhibitor or the precursor thereof. Release of MMPIs or their precursors by means of a coating provides for specific administration locally at a site where formation of fibrous tissue may occur and lowered treshold is desirable. Manufacturing of such electrodes may be easily accomplished by, e.g., dipping an ordinary electrode into a carrier composition.

Preferably, the coating itself should not impair transfer of stimulation pulses from the electrode to heart tissue. Thus, selected parts only of the electrode may be coated. Sufficient pulse transfer may also be achieved by the utilisation of a porous, thin and/or conducting coating. Appropriate pulse transfer may be provided when the carrier composition further comprises a conducting polymer. The conducting polymer may serve as a binder and/or adhesive of the coating. Suitable conducting polymers may be electronically or ionically conducting. Examples are sulfonated tetrafluorethylene copolymers (Nafion®) and polystyrene sulfonates (PSS). Additionally, conductivity may be achieved by incorporation in the carrier composition of a hydrogel.

According a second embodiment, the structure for releasing a matrix metalloproteinase inhibitor or a precursor thereof from the electrode is formed by a compartment containing a carrier composition, wherein said carrier composition embodies the matrix metalloproteinase inhibitor or the precursor thereof and wherein the compartment is arranged to allow the metalloproteinase inhibitor or the precursor thereof to pass from the compartment to the exterior of the electrode. To control the release rate of the MMPI or its precursor from the first compartment, various chemical and diffusion control mechanisms may be used as described below. It is apparent that apart from being, e.g., a container having an opening to allow such passage, the compartment may also be represented by an amount of solid or semi-solid carrier composition as such (i.e. a “plug” embodying the MMPI or precursor thereof). As an example, an MMPI or a precursor thereof may be incorporated in “steroid plugs” currently in use with certain electrodes.

According to another embodiment, the aforementioned compartment is a first compartment and the structure further includes a second compartment containing an osmotic agent, wherein the first and second compartments are separated by a flexible or moveable partition and wherein the second compartment is arranged to allow water to pass from the exterior of the electrode to the second compartment. The function of this embodiment is based on the volume increase of an osmotic agent adsorbing water from, e.g., body fluid, causing, via the moveable or flexible partition, the volume of the first compartment to decrease, as further discussed below. In this embodiment, the first compartment is preferably a container having an opening to allow the passage of MMPI or precursor thereof to the exterior of the electrode. Examples of suitable osmotic agents are sodium chloride, icodextrin, L-carnitine and its alkanoyl derivatives.

In other embodiments, the carrier composition may further include a swelling agent, capable of swelling when contacted with water. Thus, when water diffuses into the carrier composition, it swells and becomes more porous. The MMPI or precursor thereof may then diffuse more easily through the porous carrier composition in order to be released. A suitable swelling agent is polyvinyl alcohol (PVA). A swelling controlled release system is further described below.

In general, the carrier composition may further include additives as are known to those skilled in the art, of the type that physically and chemically stabilize the MMPI and the precursor thereof as well as the carrier composition as such. Those skilled in the art are capable of selecting ingredients, and their proportions, for the carrier composition in order to control the MMPI release rate. Suggestions for such ingredients are given herein. The proposed ingredients may well be combined to achieve, e.g., desirable stability, release rate and, physical (e.g. viscosity, adhesion) properties of the carrier composition. Poly(lactic-co-glycolic acid) (PLGA) is a suitable ingredient in a carrier composition for a coating.

In any embodiment of the present invention, the matrix metalloproteinase inhibitor or the precursor thereof may be releasably attached to a carrier molecule, preferably by a bond capable of degradation by hydrolysis or enzymatic degradation. Such degradation controlled release systems allows for further control of the release rate, as is further described below.

Matrix metalloproteinases (MMPs) are zinc-dependent endopeptidases. MMPs are distinguished from other endopeptidases by their dependence on metal ions as cofactors, their ability to degrade extracellular matrix, and their specific evolutionary DNA sequence. MMPs are thought to play a major role on cell behaviors such as cell proliferation, migration (adhesion/dispersion), differentiation, angiogenesis, apoptosis and host defense. More specifically, it has been found that in the case of implantable stimulation electrodes, the presence of MMPs can degrade the collagen in the cellular matrixes in the electrode/tissue interface and cause tissue destruction. This will contribute to poor tissue remodelling and result in thicker fibrous capsule formation around the electrode.

The MMPs may be inhibited by matrix metalloproteinase inhibitors (MMPIs), such as specific endogenous tissue inhibitors of metalloproteinases (TIMPs), which comprise a family of four protease inhibitors: TIMP-1, TIMP-2, TIMP-3 and TIMP-4. The structure, regulation and biological functions of TIMPs are described in Gomez, D. E. et al., Eur J Cell Biol. 1997 October; 74(2):111-122. Synthetic matrix metalloproteinase inhibitors generally contain a chelating group which binds the catalytic zinc atom at the MMP active site tightly. Common chelating groups include hydroxamates, carboxylates, thiols, and phosphinyls. Hydroxymates are particularly potent inhibitors of MMPs and other zinc-dependent enzymes, due to their bidentate chelation of the zinc atom. Other substitutents of these MMPIs are usually designed to interact with various binding pockets on the MMP of interest, making the inhibitor more or less specific for given MMPs.

The term “matrix metalloproteinase inhibitor”, “MMP inhibitor” or “MMPI” is used herein to denote any substance that is capable of inhibiting, i.e. restricting, hindering or preventing, the action of a matrix metalloproteinase (MMP). The term “inhibitor” as used herein includes agents that act indirectly by inhibiting the production of the relevant enzyme, for example an antisense molecule, as well as agents that act directly by inhibiting the enzyme activity of the relevant enzyme, such as, for example, a conventional inhibitor. An MP inhibitor may be naturally-occurring or synthetic. An MP inhibitor may be an anti-MMP antibody, either polyclonal or monoclonal. The present invention also includes the use of broad spectrum MMP inhibitors.

The inhibitory activity of a putative MMP inhibitor may be assessed by any method suitable for determining inhibitory activity of a compound with respect to an enzyme. Such methods are described in standard textbooks of biochemistry. A more detailed description of MMP inhibitors is given below.

As stated in WO 95/24921, both natural and synthetic MMP inhibitors (inhibitors of enzyme activity), including collagenase inhibitors, are known. Naturally-occurring MMP inhibitors include α₂-macroglobulin, which is the major collagenase inhibitor found in human blood. Naturally occurring MMP inhibitors are also found in tissues. The presence of tissue inhibitors of MMPs has been observed in a variety of explants and in monolayer cultures of mammalian connective tissue cells. Not only collagenase inhibitors but also inhibitors for other MMPs, for example, gelatinase and proteoglycanase are found. MMP inhibitors are generally unable to bind the inactive (zymogen) forms of the respective enzymes but complex readily with active forms. Tissue MMP inhibitors are found, for example, in dermal fibroblasts, human lung, gingival, tendon and corneal fibroblasts, human osteoblasts, uterine smooth muscle cells, alveolar macrophages, amniotic fluid, plasma, serum and the a-granule of human platelets. Synthetic collagenase inhibitors and inhibitors for other MMPs have been and are being developed. Compounds such as EDTA, cysteine, tetracycline and ascorbate are all inhibitors of collagenases but are relatively nonspecific. As indicated above, synthetic inhibitors that have defined specificity for MMPs, including collagenase inhibitors, are described in the literature. For example, U.S. Pat. Nos. 5,183,900, 5,189,178 and 5,114,953 describe the synthesis of N-[2(R)-2-(hydroxamidocarbonylmethyl)-4-methylpentanoyl]-L-tryptophan methylamide, also known as GM6001 or Galardin (trade name), and other MMP inhibitors. Other MMP inhibitors based on hydroxamic acid are disclosed in WO 90/05716, WO 90/05719 and WO 92/13831. Further synthetic MMP inhibitors and in particular collagenase inhibitors that have been developed include those described in EP-A-126,974 and EP-A-159,396 and in U.S. Pat. Nos. 4,599,361 and 4,743,587. Yet another inhibitor is BB-94, also known as Batimastat (British Bio-technology Ltd.), see for example, EP-A-276436. Disclosed in WO 90/05719 as having particularly strong collagenase inhibiting properties are [4-(N-hydroxyamino)-2R-isobutyl-3S-(thio-phenylthiomethyl)succinyl]-L-phenylalanine-N-methylamide and [4-(N-hydroxyamino)-2R-isobutyl-3S-(thiomethyl) succinyl]-L-phenylalanine-N-methylamide and in WO 90/05716 [4-(N-hydroxyamino)-2R-isobutylsuccinyl]-L-phenylalanine-N-(3-aminomethylpyridine)amide and [4-N-hydroxyamino)-2R-isobutyl-3S-methylsuccinyl]-L-phenylalanine-N-[4-(2-aminoethyl)-morpholino]amide.

The contents of WO 95/24921 and the patents, patent applications and litterature references mentioned therein, some of them rementioned above, are hereby incorporated by reference. Explicitly is incorporated any definitions of, or references to the structural or functional features of, matrix metalloproteinase inhibitors in said documents. Matrix metalloproteinase inhibitors so disclosed are suitable for use in the present invention.

In WO 02/055121 is used an MMPI which is a hydroxamic acid based collagenase inhibitor which is an oligopeptide compound, preferably of the general formula

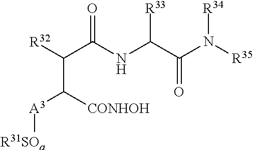

wherein:

R³¹ represents a hydrogen atom, C₁₋₆ alkyl, phenyl, thienyl, substituted phenyl, phenyl(C₁₋₆)alkyl, heterocyclyl, (C₁₋₆)alkylcarbonyl, phenacyl or substituted phenacyl group; or when a=0, R³¹ represents a group:

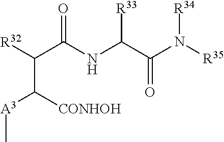

R³² represents a hydrogen atom or a C₁₋₆alkyl, C₁₋₆alkenyl, phenyl (C₁₋₆) alkyl, cycloalkyl (—C₁₋₆) alkyl or cycloalkenyl (C₁₋₆) alkyl group;

R³³ represents an amino acid side chain or a C₁₋₆alkyl, benzyl, (C₁_{_{—}}₆alkoxy)benzyl, benzyloxy (C₁_{_{—}}₆alkyl) or benzyloxbenzyl group;

R³⁴ represents a hydrogen atom or a methyl group; a is an integer having the value 0,1 or 2; and

A³ represents a C₁₋₆ hydrocarbon chain, optionally substituted with one or more C₁₋₆alkyl, phenyl or substituted phenyl groups; or a salt thereof. Most preferably the MMPI is selected from batimastat [(2R-(1(S*),2R*,3S*))-N4-hydroxy-N1-(2-(methylamino)-2-oxo-1-(phenylmethyl)ethyl)-2-(2-methylpropyl)-3-((thienylthio)methyl)butanediamide] and marimastat. Synthesis of compounds of the above general formula is described in U.S. Pat. No. 5,240,958. Batimastat itself is synthesized in Example 2 of that document.

The contents of WO 02/055121 and the patents, patent applications and literature references mentioned therein are hereby incorporated by reference. Explicitly is incorporated any definitions of, or references to the structural or functional features of, matrix metalloproteinase inhibitors in said documents. Matrix metalloproteinase inhibitors so disclosed are suitable for use in the present invention.

Modifications around the dipeptide core of a hydroxamic acid based matrix metalloproteinase inhibitor were studied in Levy, D. E. et al., J. Med. Chem. 1998, 41, 199-233. MMPIs based on the general formula

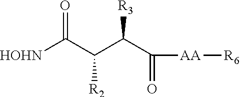

were prepared and subjected to activity studies. MMPIs prepared included the following modifications of AA and R₆. R2 was, i.a., H.

Substitutions
R3 = (R)-iBu

| AA | R6 |
|---|---|
| L-Trp | NHCH₃ |
| L-Trp | NH(CH₂)₄CH₃ |
| L-Trp | NH(CH₂)₂OH |
| L-Trp | NH(CH₂)₂NHCBZ |
| L-Trp | 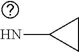 |
| L-Trp | 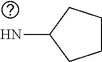 |
| L-Trp | 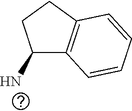 |
| L-Trp | 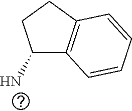 |
| L-Trp | 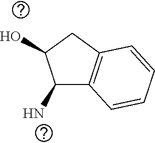 |
| L-Trp | 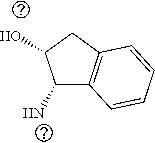 |
| L-Trp | 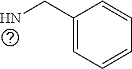 |
| L-Trp | 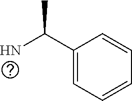 |
| L-Trp | 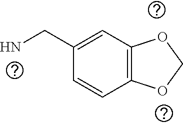 |
| L-Trp | 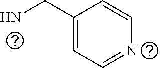 |
| L-Trp | 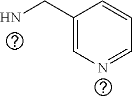 |
| L-Trp | 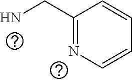 |
| L-Trp | 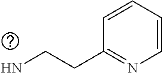 |
| L-Trp | 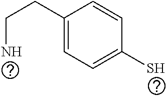 |
| L-Trp | 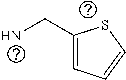 |
| L-Trp | 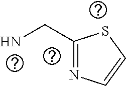 |
| L-Trp | 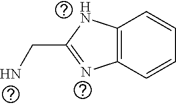 |
| L-Trp | 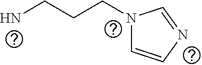 |
| L-Trp | 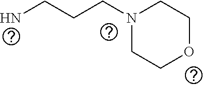 |
| L-Trp | N(CH₃)₂ |
| L-Trp | 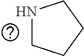 |
| L-Trp | 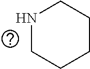 |
| L-Trp | 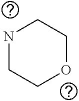 |
| D-Trp | NHCH₃ |
| L-Trp(Me) | NHCH₃ |
| L-3-Bal | NHCH₃ |
| L-1-Nal | NHCH₃ |
| L-1-Nal | 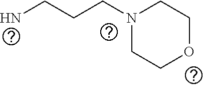 |
| L-2-Nal | NHCH₃ |
| L-2-Nal | 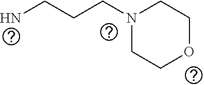 |
| L-2-Nal | 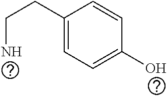 |
| L-2-Nal | 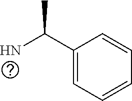 |
| L-2-Nal | 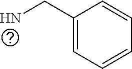 |
| L-3-Qal | NHCH₃ |
| L-8-Qal | NHCH₃ |
| L-4,4′-Bip | NHCH₃ |
| L-4,4′-Bip | 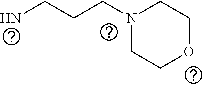 |
| L-4,4′-Bip | 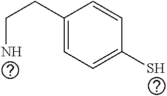 |
| L-4,4′-Bip | 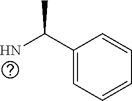 |
| L-4,4′-Bip | 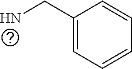 |
| L-Phe | NHCH₃ |
| L-3-Pal | 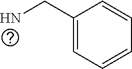 |
| L-4-Pal | NHCH₃ |
| L-4-Pal | 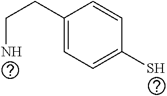 |
| L-4-Pal | 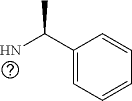 |
| L-4-Pal | 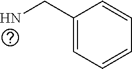 |
| L-tert-Leu | NHCH₃ |
| L-Abr | NHCH₃ |
| L-Abr | 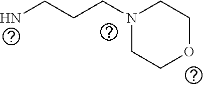 |

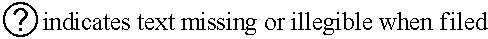

Also in Auge, F. et al., Bioorg Med Chem Lett, 2003 May 19; 13(10):1783-1786 is presented the synthesis of several analogues of galardin together with their in vitro inhibitory activity against MMP-1 and MMP-2. Compunds based on a hydrazide scaffold present potent selectivity for MMP-2 versus MMP-1.

The contents of Levy, D. E. et al., J. Med. Chem. 1998, 41, 199-233; and Auge, F. et al., Bioorg Med Chem Lett, 2003 May 19; 13(10):1783-1786 are hereby incorporated by reference. Explicitly is incorporated any definitions of, or references to the structural or functional features of, matrix metalloproteinase inhibitors in said documents. Matrix metalloproteinase inhibitors so disclosed are suitable for use in the present invention.

Doxycycline (trade name Periostat by the company CollaGenex), at subantimicrobial doses, inhibits MMP activity, and has been used as a matrix metalloproteinase inhibitor for this purpose. It is used clinically for the treatment of periodontal disease and widely available clinically. A number of rationally designed matrix metalloproteinase inhibitors, such as marimastat have shown promise in the treatment of pathologies which MMPs are suspected to be involved in. Other available MMPIs are ilomastat and trocade (Ro 32-3555), an MMP-1 selective inhibitor.

In summary, a wide choice of MMPIs is available and suitable for use in the present invention. The choice of a particular MMPI is not critical for achieving the objects of the present invention. On the contrary, the invention is based on the insight that MMPIs have desirable effects in clinical situations involving implantable cardiac stimulation electrodes. Preferred MMPIs are, however, mentioned above and/or in incorporated references.

Thus, in an embodiment of the present invention, the matrix metalloproteinase inhibitor is TIMP-1, TIMP-2, TIMP-3 or TIMP-4, or an analogue thereof. In another embodiment, the matrix metalloproteinase inhibitor is galardin, doxycyklin, batimastat, ilomastat, marimastat or trocade, or an analog thereof.

According to a second aspect of the invention, there is provided an implantable cardiac lead embodying an electrode as defined above.

According to a third aspect of the invention, there is provided a method making use of a matrix metalloproteinase inhibitor or a precursor thereof for the production of the structure for releasing the matrix metalloproteinase inhibitor or the precursor thereof from an implantable cardiac stimulation electrode. Such use may be further defined as described above.

According to a fourth aspect of the invention, there is provided a method of improving the biocompatibility of an implantable cardiac stimulation electrode, including the step of providing the electrode with s structure for releasing a matrix metalloproteinase inhibitor or a precursor thereof. Such a method may be further defined as described above.

According to a fifth aspect of the invention, there is provided a method making use of a matrix metalloproteinase inhibitor or a precursor thereof for ex vivo manufacturing of the structure for releasing the matrix metalloproteinase inhibitor or the precursor thereof from an implantable cardiac stimulation electrode, wherein said means is for lowering the chronic pacing treshold in the heart of a patient. Said use may be further defined as described above.

There is also provided a method of accomplishing a lowered chronic pacing threshold in the heart of a patient, comprising the step of implanting a cardiac lead, said lead comprising a cardiac stimulation electrode, wherein said electrode embodies a structure for releasing a matrix metalloproteinase inhibitor or a precursor thereof. Said method may be further defined as described above.

According to a sixth aspect of the invention, there is provided use of a matrix metalloproteinase inhibitor or a precursor thereof for ex vivo manufacturing of structure for releasing the matrix metalloproteinase inhibitor or the precursor thereof from an implantable cardiac stimulation electrode, wherein the structure is for prevention or reduction of formation of fibrous tissue in a patient, resulting from implantation of said electrode. Said use may be further defined as described above.

There is also provided a method of preventing or reducing formation of fibrous tissue in a patient, resulting from implantantation of a cardiac stimulation electrode, including the step of implanting a cardiac lead, the lead embodying electrode with a structure for releasing a matrix metalloproteinase inhibitor or a precursor thereof. Such a method may be further defined as described above.

### DESCRIPTION OF THE PREFERRED EMBODIMENTS

The embodiments described below are merely examples of possible means for releasing a matrix metalloproteinase inhibitor or a precursor from an implantable cardiac stimulation electrode and the present invention should not be limited thereto.

### Osmotic Pump

On example of a structure for releasing a matrix metalloproteinase inhibitor or a precursor from an implantable cardiac stimulation electrode is a so-called osmotic pump. Generally, such an osmotic pump is formed by a first compartment connected to the exterior of the electrode. The first compartment contains a carrier composition, the carrier composition in turn including the matrix metalloproteinase inhibitor or the precursor thereof. The osmotic pump further has a second compartment connected to the exterior of the electrode. The second compartment contains an osmotic agent. The osmotic agent can be mixed with a swelling agent. The first and second compartments are separated by a flexible or moveable partition.

In practice, the osmotic pump is a miniature container made from a titanium alloy, other non-degradable material or directly incorporated into components of the cardiac lead. The container is divided into two compartments, which are separated by a movable partition. One compartment contains an osmotic agent, or an osmotic agent in combination with a swelling agent, and the other an MMP inhibitor. The compartment protects and stabilizes the MMP inhibitor drug present inside. The osmotic agent attracts water from the body fluid which enters into the compartment through a semi-permeable membrane. As water enters the compartment, the volume of the osmotic and/or swelling agent increases. Since the two compartments are separated by a moveable partition, the volume of the compartment of the osmotic agent increases whereas the volume of the compartment of the MMP inhibitor is decreased. These volume changes cause delivery of the MMP inhibitor from an orifice in the compartment of the MMP inhibitor.

### Swelling Controlled Release System

Another example of a structure for releasing a matrix metalloproteinase inhibitor or a precursor from an implantable cardiac stimulation electrode is represented by a swelling controlled release system. Generally, this means that the MMP inhibitor or the prodrug thereof is present in an agent capable of swelling when contacted with water. Said agent may be present as a coating on an electrode or in a compartment of an electrode.

Thus, the MMP-inhibitor is dispersed into a matrix consisting of a polymer that is stiff or glassy when dry, but swells when placed in an aqueous environment. The polymer can be, but is not limited to, a polyvinyl alcohol (PVA) which is hydrophilic and swells easily by absorbing water. When water diffuses into the polymer/drug matrix, it swells and becomes more porous. The MMP inhibitor is now capable of diffusing through the porous phase to be released to the environment. Swelling controlled release system is described in J Control Release. 2000 Jul. 31; 68(1):115-20 and J Control Release. 2000 Feb. 3; 63(3):297-304.

Chemical or Diffusion Controlled Release from a MHP Inhibitor “Plug” on the Electrode

The electrode design includes a compartment wherein the MMP inhibitor or the prodrug thereof can be stored. To control the release rate of the MMP-inhibitor from the compartment, different chemical or diffusion control mechanisms can be used.

Mechanism A, pendant-chain system delivery: Pendant-chain systems have degradable linkages that release drug molecules upon exposure to water. MMP inhibitor is chemically linked directly, or via a spacer to a polymer backbone. The backbone can be biodegradable or non-degradable. The spacer undergoes hydrolyzation or enzymatic degradation faster than the degradation of the polymer backbone (if it is degradable). The MMP inhibitor is released when the chemical bonds directly to the backbone or the chemical bonds to the spacer are broken by hydrolyzation or enzymatic degradation. As an example, the backbone can be a hydroxypropyl methacrylate (HPMA) copolymer with adriamycin linked by Gly-Phe-Leu-Gly, as described by Soyer et. al., Adv. Drug Del. Rev., 2 (1996) 81-106.

Mechanism B, membrane-controlled delivery: The MMP inhibitor is contained in a core, which is surrounded by a polymer membrane. The MMP inhibitor it is released by diffusion through this rate-controlling membrane. The MMP inhibitor can also be loaded into a polymer matrix which is surrounded by the membrane. Examples of suitable membranes are ethylene vinyl acetate (EVA), ethylene vinyl acetate copolymer (EVAc), silicone rubber (e.g. Silastic, Dow Corning), ethyl cellulose. Membrane-controlled delivery is further described in Pharm Res. 1998 August; 15 (8) :1238-43 and J. Pharm. Pharmaceut. Sci, 8(1):26-38, 2005.

Mechanism C, monolithic drug delivery: The MMP inhibitor is uniformly dispersed or dissolved in a polymer or co-polymer. The MMPI is released to the surrounding by diffusion from the polymer. The drug delivery system can be composed of Eudragit RL 100 (ammonio methacrylate copolymer) and polyvinyl pyrrolidone (PVP) (in various ratios) along with different amount of of MMP inhibitor, plasticizer, polyethylene glycol-400 and dimethyl sulfoxide as penetration enhancer. Monolithic drug delivery is further described in AAPS Pharm Sci Tech 2006; 7(1) Article 6.

Mechanism D, biodegradable drug delivery system The MMP inhibitor is dispersed into a polymer or co-polymer which is eroded and thus releases the MMP inhibitor. The polymers or co-polymers used in the formulation and fabrication erode (with or without changes to the chemical structure) or degrade (breakdown of the main chain bonds) as a result of the exposure to chemicals (water) or biologicals (enzymes). The drug molecules, which are initially dispersed in the polymer, are released as the polymer starts eroding or degrading. The four most commonly used biodegradable polymers in such drug delivery systems are poly(lactic acid), poly(lactic-co-glycolic acid), polyanhydrides, poly(ortho esters), and poly(phosphoesters). Biodegradable drug delivery system are further described in Investigative Ophthalmology & Visual Science, May 1994, Vol. 35, No. 6; Crit Rev Ther Drug Carrier Syst. 1984;1(1):39-90; and J Control Release. 1998 Mar. 2; 52(1-2):179-89.

Chemical or Diffusion Controlled Release from a MMP Inhibitor Coating on the Electrode

Mechanisms A-D as described above may also be utilized for delivery of MMP inhibitors from a coating on the electrode. However, such a coating should preferably not interfere with the electric or ionic contact between the electrode and heart tissue. Accordingly, it is preferred to use a conducting coating. Modification of a coating so as to achieve electrical conductivity is described in the example below.

### EXAMPLE

Production of a Cardiac Electrode Coated with Conducting Polymer Modified Hydrogel Loaded with Matrix Metalloproteinase Inhibitor Imbedded in PLGA Particles

### Oil-in-Water (O/W) Emulsion/Solvent Evaporation Method

800 mg Poly(lactic-co-glycolic acid), PLGA, is dissolved in 15 ml dichloromethane. Approximately 200 mg matrix metalloproteinase inhibitor, MMPI, is dissolved in 15 ml acetone (or other suitable solvent). MMPI in solvent is added to PLGA in dichloromethane to form an oil-phase. The oil-phase is added drop by drop to an aqueous solution containing 5% poly(vinyl alcohol), PVA. An oil-in-water type emulsion is formed with a sonicator (approximately 10 min with a constant power output of 60 W). The organic solvent is evaporated by gently stirring the solution at room temperature for 12 h. The unreacted drug and PVA residue is washed three times with deionised water. Nanoparticles are collected with the aid of a centrifuge for 1 hour. A fine power is obtained by lyophilization. Biomaterials 27, 3031 (2006).

Production of Coating of Alginate Hydrogel Loaded with Matrix Metalloproteinase Inhibitor PLGA

MVG alginate powder is dissolved in double-distilled water, while mixing with a magnetic stirrer, to a concentration of 1-3 wt %. Nano-particles with imbedded MMPI are added to the alginate solution to desired amount. The solution is gelled by ionic cross-linking with 0.5 M CaCl₂. The alginate hydrogel with imbedded MMPI particles is deposited on the electrode surface by dipping.

### Modification of the Hydrogel Coating by Deposition of Electrically Conducting Polymer

After coating of the electrode with the hydrogel loaded with matrix metalloproteinase inhibitor PLGA, it is an option to include electrically conducting polymers within the hydrogel in order to modify the coating for enhanced stimulation/sensing characteristics. Conducting polymers can be electrochemically synthesized inside a hydrogel support matrix, by galvano- or potentiostatic/dynamic methods.

The conducting polymer can be deposited in a three-electrode electrochemical cell, with a Pt-mesh as a counter electrode, a calomel electrode as a reference electrode and the hydrogel (comprising matrix metalloproteinase inhibitor PLGA) covered electrode as a working electrode. A monomer solution is used as the electrolyte. Depending on the type of polymer desired, a suitable monomer solution should be selected. For deposition of polystyrene sulfonate (PSS) a 40 ml electrolyte consisting of 0.2 M pyrrole monomer, 0.2 M PSS and 4 ml of 1 M CaCl₂ is used. To prevent oxidation of the monomer, the electrolyte is purged with N₂ for 10 min before use. The polymer is deposited at a galvanic current density of 4.8 mA/cm².

Although modifications and changes may be suggested by those skilled in the art, it is the intention of the inventor to embody within the patent warranted heron all changes and modifications as reasonably and properly come within the scope of her contribution to the art.

1.-22. (canceled)

23. An implantable cardiac stimulation electrode comprising:
an electrode body configured for in vivo implantation in a patient to deliver electrical stimulation pulses to tissue surrounding said electrode body;
a releasable material selected from the group consisting of a matrix metalloproteinase inhibitor and a matrix metalloproteinase inhibitor precursor; and
a release structure carried by said electrode body, said release structure containing said releasable material and being configured to allow release of said releasable material into said tissue following implantation of said electrode body.

24. An implantable cardiac stimulation electrode as claimed in claim 23 wherein said release structure comprises a coating comprising a carrier composition on at least a portion of a surface of said electrode body, said carrier composition comprising said releasable material.

25. An implantable cardiac stimulation electrode as claimed in claim 24 wherein said carrier composition further comprises a conducting polymer.

26. An implantable cardiac stimulation electrode as claimed in claim 24 wherein said carrier composition further comprises a swelling agent that swells when in contact with water.

27. An implantable cardiac stimulation electrode as claimed in claim 23 wherein said release structure comprises a compartment in said electrode body containing a carrier composition, said carrier composition comprising said releasable material, said compartment being configured to allow said releasable material to pass from said compartment to an exterior of said electrode body.

28. An implantable cardiac stimulation electrode as claimed in claim 27 wherein said compartment is a first compartment, and wherein said release structure further comprises a second compartment in said electrode body containing an osmotic agent, and a flexible or movable partition separating said first and second compartments, said second compartment being configured to allow water to pass from an exterior of said electrode body into said second compartment.

29. An implantable cardiac stimulation electrode as claimed in claim 28 wherein said carrier composition further comprises a swelling agent that swells when in contact with water.

30. An implantable cardiac stimulation electrode as claimed in claim 23 comprising a carrier molecule releasably attached to said releasable material by a bond selected from the group consisting of bonds degradable by hydrolysis and enzymatically degradable bonds.

31. An implantable cardiac stimulation electrode as claimed in claim 23 wherein said matrix metalloproteinase inhibitor is selected from the group consisting of TIMP-1, TIMP-2, TIMP-3, TIMP-4, and analogs thereof.

32. An implantable cardiac stimulation electrode as claimed in claim 23 wherein said matrix metalloproteinase inhibitor is selected from the group consisting of galardin, doxycyklin, bactimastat, ilomastat, marimastat, trocade, and analogs thereof.

33. An implantable cardiac lead comprising:
a lead body’
an electrode body carried by said lead body, said electrode body and said lead body being configured for in vivo implantation in a patient to deliver electrical stimulation pulses to tissue surrounding said electrode body;
a releasable material selected from the group consisting of a matrix metalloproteinase inhibitor and a matrix metalloproteinase inhibitor precursor; and
a release structure carried by said electrode body, said release structure containing said releasable material and being configured to release said releasable material into said tissue after implantation of said lead body and said electrode body.

34. A method for manufacturing an implantable cardiac electrode lead, comprising the steps of:
providing an electrode body on a lead body configured for in vivo implantation in a patient; and
providing a release structure on said electrode body containing a releasable material, and configuring said release structure to release said releasable material into tissue surrounding said electrode body following implantation of said electrode lead and said electrode body; and
selecting said releasable material from the group consisting of matrix metalloproteinase inhibitors and matrix metalloproteinase inhibitor precursors.

35. A method for in vivo delivery of electrical stimulation to cardiac tissue, comprising the steps of:
implanting, in a patient, a lead body carrying an electrode body to position said electrode body at a location that causes said electrode body to interact with cardiac tissue;
delivering electrical stimulation to said cardiac tissue via said lead body and said electrode body;
prior to implanting said lead body and said electrode body, providing said electrode body with a release structure that contains a release material, and configuring said release structure to release said releasable material therefrom into said cardiac tissue after implantation of said lead body and said electrode body; and
selecting said releasable material from the group consisting of matrix metalloproteinase inhibitors and matrix metalloproteinase inhibitor precursors.

36. A method as claimed in claim 35 comprising forming said release structure as an osmotic pump in said electrode body, and releasing said releasable material from said release structure after implantation of said lead body and said electrode body by operation of said osmotic pump.
